(19) United States
(12) Patent Application Publication
(10) Pub. No.: US 2023/0140863 A1
(43) Pub. Date: May 11, 2023

(54) SYSTEMS AND METHODS FOR OPERATING AN IMPLANTABLE MEDICAL DEVICE BASED UPON A SENSED PHYSICAL ACTION

(71) Applicant: INSPIRE MEDICAL SYSTEMS, INC., Golden Valley, MN (US)

(72) Inventors: Kevin Verzal, Lino Lakes, MN (US); Nicholas Nesbitt, Minneapolis, MN (US); John Rondoni, Plymouth, MN (US); David Dieken, Minneapolis, MN (US); Christopher Thorp, Minneapolis, MN (US)

(73) Assignee: INSPIRE MEDICAL SYSTEMS, INC., Golden Valley, MN (US)

(21) Appl. No.: 16/978,283

(22) PCT Filed: Jul. 24, 2020
(86) PCT No.: PCT/US2020/043405

§ 371 Date: Sep. 4, 2020

Related U.S. Application Data
(60) Provisional application No. 62/878,602, filed on Jul. 25, 2019.

(51) Int. Cl. A61B 5/00; A61N 1/36

(52) U.S. Cl. CPC A61B 5/4815; A61N 1/36078

(57) ABSTRACT

A system and/or method to control operation of an implantable medical device in response to sensed occurrence of a designated physical action intentionally performed by the patient.

### BACKGROUND

Many implantable medical devices make use of an external device (e.g., remote control). The external device may be used to activate therapy, adjust therapy, transfer data, and/or report diagnostics. The external device is an additional component to the system, and is prone to typical problems (e.g., patient forgets or loses the external device, the external device is damaged, battery depletion, etc.). As they currently exist, external devices and wireless data transfer also pose a cybersecurity risk.

### BRIEF DESCRIPTION OF THE DRAWINGS

FIG. 1 is a block diagram schematically illustrating a patient therapy system in accordance with principles of the present disclosure.

FIG. 2 is a block diagram schematically representing an implantable medical device useful with the patient therapy system of FIG. 1 and in a partially assembled state.

FIG. 3 is a block diagram schematically representing the implantable medical device of FIG. 2 in an assembled state and an implantable sensor in accordance with principles of the present disclosure assembled to the implantable medical device.

FIG. 4 is a block diagram of portions of another patient therapy system in accordance with principles of the present disclosure and including an implantable sensor carried within the housing of an implantable pulse generator assembly.

FIG. 5 is a block diagram schematically representing an example sensor lead useful with the patient therapy system of FIG. 1 and including a stimulation electrode and an implantable sensor.

FIG. 6 is a block diagram schematically representing an example system including an implantable pulse generator assembly and a separate implantable sensor in accordance with principles of the present disclosure.

FIG. 7 is a block diagram schematically illustrating portions of another patient therapy system in accordance with principles of the present disclosure.

FIG. 8A is a block diagram schematically representing an example control portion.

FIG. 8B is a diagram schematically representing at least some examples different modalities of the control portion of FIG. 8A.

FIG. 8C is a block diagram schematically representing an example user interface.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to systems and methods for controlling at least some operations of an implantable medical device implanted within a patient based upon the sensed intentional performance of a designated physical action by the patient. In some embodiments, one or more sensors implanted in the patient are utilized to sense or detect the intentional performance of a designated physical action by the patient. In some embodiments, the operations of the implantable medical device that are controlled in response to the sensed intentional performance of a designated physical action relate to an interface (e.g., wireless communications) between the implantable medical device and an external device. In some examples, the operations of the implantable medical device that are controlled in response to the sensed intentional performance of a designated physical action relate to an operational mode of the implantable medical device. In some examples, the systems of the present disclosure are configured and used for sleep disordered breathing (SDB) therapy, such as obstructive sleep apnea (OSA) therapy. However, in other examples, the system is used for other types of therapy, including, but not limited to, other types of neurostimulation therapy. In some embodiments, such other implementations include therapies, such as but not limited to, central sleep apnea, complex sleep apnea, cardiac disorders, and respiratory disorders.

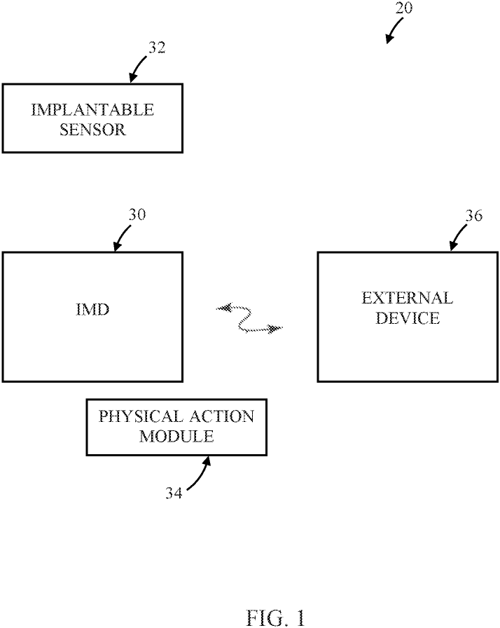

One example of a patient therapy system 20 in accordance with principles of the present disclosure is schematically represented in FIG. 1. The patient therapy system 20 includes an implantable medical device (IMD) 30, one or more implantable sensors 32, a physical action module or handler 34, and an optional external device 36. Details on the various components are provided below. In general terms, the IMD 30 is configured for implantation into a patient, and is configured to provide and/or assist in the performance of therapy to the patient. The implantable sensor 32 can assume various forms, and is generally configured for implantation into a patient and to at least sense a parameter indicative of one or more physical actions performed by the patient. The implantable sensor 32 can be carried by the IMD 30, can be connected to the IMD 30, or can be a standalone component not physically connected to the IMD 30. The physical action module 34 receives information from the implantable sensor 32 and is programmed (or is connected to a separate module that is programmed) to recognize or identify or determine that the patient has performed a designated physical action based, at least in part, upon information from the implantable sensor 32. In some embodiments, the physical action module 34 is programmed (or is connected to a separate module that is programmed) to effect (or not effect) one or more control routines or the like relating to operation of the system 20 in response to a determination that the patient has performed a designated physical action. As described below, the physical action module 34 can be incorporated by the IMD 30 (e.g., installed into a software application operated by the IMD 30), or can reside, either partially or entirely, with other components of the system 20. Where provided, the external device 36 can wirelessly communicate with the IMD 30, and is operable to control operations of the IMD 30 as described below. In other embodiments, the external device 36 can be omitted (e.g., the IMD 30, the implantable sensor 32 and the physical action module 34 perform one or more of the operations described below without the need for an external device).

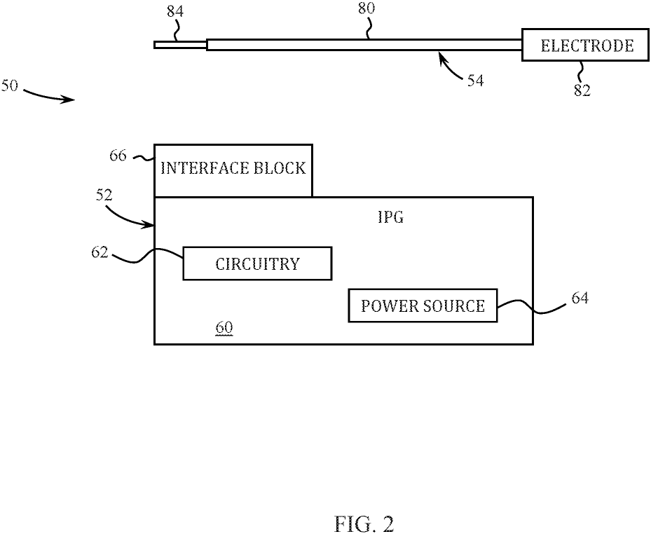

FIG. 2 is a block diagram schematically representing one example of an IMD 50 useful with the systems and methods of the present disclosure, for example as the IMD 30 of the system 20 of FIG. 1. The IMD 50 can include an implantable pulse generator (IPG) assembly 52 and a stimulation lead 54. The IPG assembly 52 can include a housing 60 containing circuitry 62 and a power source 64 (e.g., battery), and an interface block or header-connector 66 carried or formed by the housing 60. The housing 60 is configured to render the IPG assembly 52 appropriate for implantation into a human body, and can incorporate biocompatible materials and hermetic seal(s). The circuitry 62 can include circuitry components and wiring apparent to one of ordinary skill appropriate for generating desired stimulation signals (e.g., converting energy provided by the power source 64 into a desired stimulation signal), for example in the form of a stimulation engine. In some embodiments, the circuitry 62 can include telemetry components for communication with external devices as is known in the art. For example, the circuitry 62 can include a transmitter that transforms electrical power into a signal associated with transmitted data packets, a receiver that transforms a signal into electrical power, a combination transmitter/receiver (or transceiver), an antenna, etc.

In some embodiments, the stimulation lead 54 includes a lead body 80 with a distally located stimulation electrode 82. At an opposite end of the lead body 80, the stimulation lead 54 includes a proximally located plug-in connector 84 which is configured to be removably connectable to the interface block 66 (e.g., the interface block 66 can optionally include or provide a stimulation port sized and shaped to receive the plug-in connector 84 as is known in the art).

In general terms, the stimulation electrode 82 can optionally be a cuff electrode, and can include some non-conductive structures biased to (or otherwise configurable to) releasable secure the stimulation electrode 82 about a target nerve. Other formats are also acceptable. Moreover, the stimulation electrode 82 can include an array of electrode bodies to deliver a stimulation signal to a target nerve. In some non-limiting embodiments, the stimulation electrode 82 can comprise at least some of substantially the same features and attributes as described within at least U.S. Pat. No. 8,340,785 issued Dec. 25, 2012 and/or U.S. Pat. Application Publication No. 2011/0147046 published Jun. 23, 2011 the entire teachings of each of which are incorporated herein by reference in their entireties.

In some examples, the lead body 80 is a generally flexible elongate member having sufficient resilience to enable advancing and maneuvering the lead body 80 subcutaneously to place the stimulation electrode 82 at a desired location adjacent a nerve, such as an airway-patency-related nerve (e.g. hypoglossal nerve). In some examples, such as in the case of obstructive sleep apnea, the nerves may include (but are not limited to) the nerve and associated muscles responsible for causing movement of the tongue and related musculature to restore airway patency. In some examples, the nerves may include (but are not limited to) the hypoglossal nerve and the muscles may include (but are not limited to) the genioglossus muscle. In some examples, lead body 80 can have a length sufficient to extend from the IPG assembly 52 implanted in one body location (e.g. pectoral) and to the target stimulation location (e.g. head, neck). Upon generation via the circuitry 62, a stimulation signal is selectively transmitted to the interface block 68 for delivery via the stimulation lead 54 to such nerves.

Returning to FIG. 1, the implantable sensor 32 can assume various forms appropriate for implantation into a human patient, and generally includes a sensor component in the form of or akin to a motion-based transducer. In some embodiments, the motion-based transducer sensor component of the implantable sensor 32 can be or include an accelerometer (e.g., a multi-axis accelerometer such as a three-axis accelerometer), a gyroscope, a pressure sensor, etc., as is known in the art. Regardless of an exact form, the sensor component of the implantable sensor 32 is capable of sensing, amongst other things, information indicative of a patient performing one or more of the physical actions described below. As a point of reference, while information generated by the implantable sensor 32 is signaled to and acted upon by the physical action module 34 as described below, information from the implantable sensor 32 can be utilized by other modules or engines (e.g., a therapy manager module that manages therapy delivered to the patient by the IMD 30 as described below).

The implantable sensor 32 can be connected to the IMD 30 in various fashions. For example, and with additional reference to the IMD 50 of FIG. 2, the implantable sensor 32 can include a lead body carrying the motion-based transducer sensor element at a distal end, and a plug-in connector at proximal end. The plug-in connector can be connected to the interface block 66 (e.g., the interface block 66 can include or provide a sense port sized and shaped to receive the plug-in connector of the implantable sensor 32), and the lead body extended from the IPG assembly 52 to locate the sensor element at a desired anatomical location. Accordingly, physical action-related information sensed via the motion-based transducer element is transmitted, via the interface block 66, to the circuitry 62.

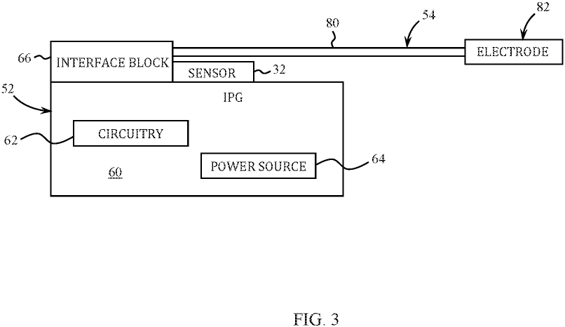

Alternatively, and as reflected by the block diagram of FIG. 3, the implantable sensor 32 can be physically coupled to the interface block 66, and thus carried by the IPG assembly 52 (e.g., the implantable sensor 32 can be considered a component of the IMD 50). Among other features, this optional arrangement may eliminate tunneling and/or other surgical steps ordinarily associated with placing sensing leads wihtin a patient, as well as promote long term stability and ease securing the implantable sensor 32 because it occurs in conjunction with securing the IPG assembly 52. In some embodiments, the physical coupling of the implantable sensor 32 relative to the IPG assembly 52 is performed prior to implantation of those components.

In some embodiments, in order for the motion-based transducer-type implantable sensor 32 to fit on top of (e.g. next to) the housing 60 of the IPG assembly 52, a housing of the implantable sensor 32 has a size and shape that can maintain the motion-based transducer sensor component in a fixed orientation relative to the IPG assembly 52. This arrangement facilitates achieving and maintaining a proper orientation of the multiple orthogonal axes of the motion-based transducer sensor component relative to various axes of the patient’s body, such as an anterior-posterior axis.

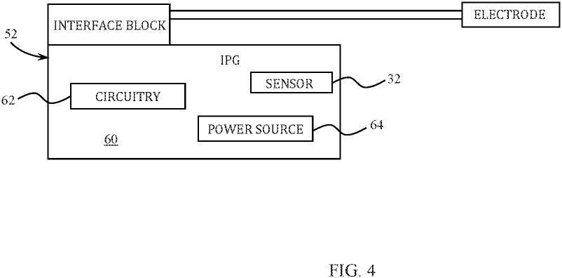

In related embodiments, and as reflected by the block diagram of FIG. 4, the implantable sensor 32 (and in particular the motion-based transducer sensor component as described above) can be incorporated into a structure of the interface block 66 or into a structure of the housing 60. With these and similar configurations, the sensor component of the implantable sensor 32 is electronically connected to the circuitry 62 within the housing 60 or other enclosure of the IPG assembly 52.

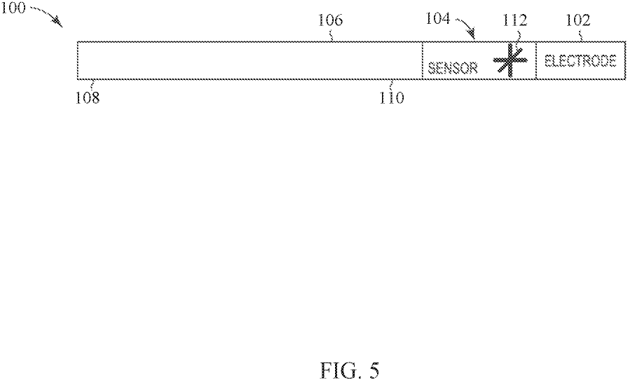

In yet other embodiments, the implantable sensor 32 can be incorporated into a structure of the stimulation lead 54. For example, FIG. 5 is a block diagram schematically representing a stimulation lead 100 including a stimulation electrode 102 and a motion-based transducer sensor 104 (akin to the implantable sensor 32 described above), according to one example of the present disclosure. In some examples, the stimulation lead 100 comprises at least some of substantially the same features and attributes as the lead 54 in FIG. 2, except for additionally including the motion-based transducer sensor 104. As shown in FIG. 5, the lead 100 comprises a lead body 106 having a proximal end 108 configured to be removably connectable to a port of an IPG assembly (e.g., the interface block 68 of the IPG assembly 52 of FIG. 2) and an opposite distal end 110 at which the motion-based transducer sensor 104 and the electrode 102 are mounted. In some examples, the sensor 104 is located closer to the distal end 106 than the proximal end 104 of the lead 100 without necessarily being at the distal end 110 of the lead body 106. In some optional embodiments, a portion of the lead 100 at which the motion-based transducer sensor 104 is located includes a mechanism (e.g., a rotatable sleeve) to enable selective rotation of the motion-based transducer sensor 104, which in turns enables adopting a desired orientation of the different axes 112 of the motion-based transducer sensor 104 (for example where the motion-based transducer sensor 104 is a multi-axis accelerometer).

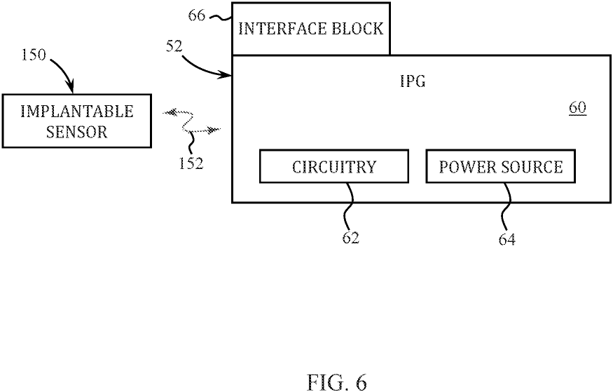

In yet other embodiments, the implantable sensor 32 can be wirelessly connected to the IMD 30. For example, FIG. 6 is a block diagram schematically representing a system including the IPG assembly 52 as described above and a separate implantable sensor 150, according to one example of the present disclosure. The implantable sensor 150 comprises at least some of substantially the same features and attributes as the previously described implantable sensors including the motion-based transducer sensor component, except for the lack of physical coupling of the implantable sensor 150 relative to IPG assembly 52; instead, the implantable sensor 150 is electrically and communicatively coupled wirelessly relative to the IPG assembly 52. With this in mind, the interface block 66 need not provide a sense port for the implantable senor 150 or the sense port can be used for a second sensor (not shown). In some embodiments, the circuitry 62 of IPG the assembly 52 and circuitry (not shown) of the implantable sensor 150 communicate via a wireless communication pathway 152 according to known wireless protocols, such as Bluetooth, NFC, 802.11, etc. with each of the circuitry 62 and the implantable sensor 150 including corresponding components for implementing the wireless communication pathway 152. In some examples, a similar wireless pathway is implemented to communicate with devices external to the patient’s body for at least partially controlling the implantable sensor 150 and/or the IPG assembly 52, to communicate with other devices (e.g. other sensors) internally within the patient’s body, or to communicate with other sensors external to the patient’s body as described in greater detail below.

Returning to FIG. 1, regardless of a format of the implantable sensor 32, the physical action module 34 is programmed to perform one or more operations as described below based upon information from the implantable sensor 32 (e.g., the implantable sensor 32 output is an input to the physical action module 34). In general terms, the physical action module 34 is programmed to recognize or determine or identify, based at least in part upon information signaled by the implantable sensor 32, the occurrence of one or more designated physical actions as having been performed by the patient. The physical action module 34 is further programmed (or signals information implicating the sensed occurrence of a designated physical action to another module or engine that is programmed) to perform one or more operations or routines relating to control of the system 20 (e.g., controlling operations of the IMD 30, the implantable sensor 32, the external device 36, an attempted communication link between the IMD 30 and the external device 36, etc.). In other embodiments, the physical action module 34 (or the logic akin to the physical action module 34 as described below) can be incorporated into a distinct module or engine programmed to perform certain tasks (e.g., logic of the physical action module 34 as described below can be part of a telemetry control engine and utilized as part of a secure telemetry connection protocol). The physical action module 34 (or the algorithms as described below) can reside partially or entirely with the IMD 30 (e.g., the circuity 62 (FIG. 2)), partially or entirely with the external device 36, or partially or entirely with a separate device or component (e.g., the cloud, etc.).

### Designated Physical Actions

As implicated by the above, the physical action module 34 is programmed or designed (e.g., appropriate algorithms) to recognize or identify or detect occurrence of at least one designated physical action based upon information from the implantable sensor 32. The designated physical actions of the present disclosure can assume a wide variety of forms, and can be detected by the implantable sensor 32 being implanted at a corresponding implant location of the patient’s anatomy (e.g., pectoralis, neck, abdomen, etc.). In some embodiments, the designated physical action relates to a defined motion of the patient’s body (or a portion of the patient’s body). For example, the designated physical action can include, but is not limited to, jumping up and down, standing on tip toes, spinning around, raising and/or lowering a particular limb (e.g., raising an arm), walking, etc., and/or a combination (simultaneously or in a prescribed order) of two or more body motions. In some embodiments, the designated physical action relates to a manual activity. For example, the designated physical action can include, but is not limited to, one or more taps on the patient’s body in or near a region of the sensor component of the implantable sensor 32 in a prescribed manner (e.g., a prescribed pattern of taps over a predetermined time interval, such as tapping more than once or tapping in a specific pattern (e.g., two times in less than a time interval, three times in less than a time interval), etc.). Some non-limiting examples of discreet motions (e.g., actions that can be performed by the patient in public without others noticing) that can serve as (or as part of) the designated physical actions of the present disclosure include arm wiggle, pectoralis flex, shoulder roll, shoulder or hip wiggle, jaw rotation, tongue wiggle, head motion (e.g., left-and-right, up-and-down), pelvic floor contractions, etc. Some non-limiting examples of non-discreet motions that can serve as (or as part of) the designated physical actions of the present disclosure include tapping, sitting up, lying down, standing up, bending over, arm rotation, etc. In some embodiments, the designated physical action relates to a posture of the patient. In at least this context, the term “posture” refers at least to identifying whether a patient is in a generally vertical position or a lying down position, such as a supine position, a prone position, a left side position (e.g., left lateral decubitus), a right side position (e.g., right lateral decubitus), etc. In some instances, the term “posture” may sometimes be referred to as “body position”. For example, the designated physical action can include, but is not limited to, the patient assuming an upright or vertical position (alone or in combination with some other physical action, such as a body motion or manual activity).

In addition to, or as an alternative to, the above, in some embodiments the designated physical action relates to or incorporates operation of an external device, such as the external device 36. For example, an external device can be provided that is configured and programmed to generate a force or motion or other “signal” when prompted by a user at levels that can be perceived or sensed by the sensor component of the implantable sensor 32. In some non-limiting embodiments, the external device can be configured to vibrate at a pre-determined frequency or within a pre-determined frequency range (e.g., a “vibration mode” commonly available with many handheld electronic devices); when located along the patient’s body in close proximity to the sensor component of the implantable sensor 32 and prompted to operate, the external device vibrates and this vibration is sensed by the implantable sensor 32 at levels or frequencies that can be “recognized” by the physical action module 34 as described below. In other words, the frequency or pattern of vibration can be used to send distinct communications to the implantable sensor 32, and thus the physical action module 34. The patterns of vibration may take the form of analog or digital modulation such as amplitude shift keying, on/off keying, frequency shift keying, and the like in order to transmit information from the external device to the implantable sensor 32. The physical action module 34, in turn, can be programmed or designed to decide the modulated signal(s).

The designated physical actions of the present disclosure can assume other forms not specifically delineated above. In more general terms, a designated physical action of the present disclosure can be any intentional physical act performed by a patient that can be sensed or be determined to have occurred based, at least in part, upon information provided by an implanted motion-based transducer sensor component. One or more of the designated physical action(s) acted upon by the physical action module 34 can include a combination of two or more of the physical actions mentioned above in a prescribed sequence and/or within a designated time period.

Logic (e.g., algorithms) embodied by the physical action module 34 can recognize or identify or detect occurrence of a particular designated physical action in various manners. In some embodiments, a particular designated physical action can be recognized or identified by a relatively straightforward algorithm that references only information from the implantable sensor 32 (e.g., if the signal from the implantable sensor 32 includes characteristic X, then the corresponding designated physical action is identified or deemed to have occurred). In other embodiments, a particular designated physical action can be recognized or identified with reference to the information from the implantable sensor 32 along with information from other data sources (e.g., a particular designated physical action is identified or deemed to have occurred when information from the implantable sensor 32 satisfies a first predetermined criteria and information from a second sensor (e.g., another implantable sensor carried by the IMD 30 such as a heart rate monitor, respiration sensor, etc.) satisfies a second predetermined criteria; when information from the implantable sensor 32 satisfies a rule or criteria at a certain time (or range of times) of day, etc.). In yet other embodiments, a particular designated physical action can be recognized or identified with reference to information from the implantable sensor 32, information for other data sources, and a probabilistic decision tree/algorithm (e.g., modeling or artificial intelligence or artificial learning). For example, one or more data sources (including information from the implantable sensor 32) can be employed in a probabilistic decision model to recognize or identify a distinction between a physical action of the patient laying down while tapping on his/her skin in or near a region of the sensor component of the implantable sensor 32 and a physical action of the patient standing while tapping on his/her skin. With these and related embodiments, then, the physical action module 34 is programmed to evaluate the probability that a particular designated physical action has occurred or is occurring, and deem or decide that the particular designated physical action has occurred (for purposes of initiating an operational control routine as described below) when the evaluated probability is acceptably high enough (e.g., likelihood of occurrence is deemed to be 80% or greater).

In some embodiments, the physical action module 34 is programmed to recognize or identify or detect one designated physical action. When information signaled from the implantable sensor 32 corresponds with identification rules or a “recipe” programmed to the physical action module 34, the physical action module 34 can recognize or identify or detect that the designated physical action has occurred (e.g., the physical action module 34 can be programmed to review information signaled from the implantable sensor 32 for a series of up-and-down motions over a short period of time and, upon recognizing such occurrence, determine or identify or detect that a designated physical action of the patient jumping up and down has occurred). In other embodiments, the physical action module 34 is programmed to recognize or identify or detect two or more, different designated physical actions. For example, the physical action module 34 can include or make reference to a library of different designated physical actions each with defined parameters that can be implicated by information from the implantable sensor 32. The physical action module 34 can continuously or periodically poll information from the implantable sensor(s) 32, comparing the received information with the defined parameters stored in the library and identifying occurrence of a particular designated physical action when the signaled information matches (or is deemed to be sufficiently close to) the defined parameters of the corresponding designated physical action. In some examples, the “designated physical action” (the occurrence of which prompts an operation) includes two or more, separately identifiable actions that must occur simultaneously or in rapid succession. For example, tapping on the patient’s body in a region of the implantable sensor 32 (e.g., a designated number of times) simultaneously with and/or right before or right after at least one of assuming a particular body posture, raising an arm, walking, spinning, or jumping. Alternatively or in addition, the physical action module 34 can be prompted by another module or engine to monitor for a particular designated physical action; under these and similar circumstances, the physical action module 34 is programmed, in response to the request, to compare current information signaled from the implantable sensor 32 (and perhaps other data sources) with the defined parameters corresponding to the particular designated physical action as stored in the library and signal the results to the requesting module or engine.

### Operational Control

A variety of control operations can be effected or performed in response to a determination that a designated physical action has been performed by the patient in accordance with principles of the present disclosure. In some embodiments, the physical action module 34 can be programmed to perform, or prompt the performance of, a particular control operation or routine. In other embodiments, another module or engine can be programmed to perform, or prompt the performance of, a particular control operation or routine in response to information from the physical action module 34 indicating that a particular designated physical action has been performed by the patient. Regardless, in some embodiments, the control operation or routine can relate to or include altering a mode of operation of the IMD 30 (e.g., switching from a current mode to a target mode), for example a mode of operation of the IPG assembly 52 (FIG. 2).

For example, and with additional reference to FIG. 2, the IPG assembly 52 can be configured and/or programmed to operate in different modes (e.g., a sleep or inactive mode in which minimal power is being consumed and no stimulation therapy is provided, a wakeup mode in which the IPG assembly 52 transitions from the sleep mode and prepares necessary connections and programming for pairing with an external device (pairing mode) and/or delivering stimulation therapy (active mode), an active mode in which the IPG assembly 52 delivers stimulation therapy at programmable levels, a safe mode in which the IPG assembly 52 delivers stimulation therapy at minimal levels, etc.). In some embodiments, the operational control module(s) or engine(s) necessary to effect one or more of the operational modes is provided with the IPG assembly 52 (e.g., part of the circuitry 62); in other embodiments, the operational control module(s) or engine(s) are provided with an auxiliary device (e.g., the external device 36) that operates to prompt the IPG assembly 52 to operate in a particular mode.

With some non-limiting embodiments in which the IPG assembly 52 includes at least one operational control module or engine, the systems and methods of the present disclosure can include the IPG assembly 52 being programmed to automatically initiate or operate in a particular operational mode upon determined occurrence of a corresponding designated physical action. By way of non-limiting example, the IPG assembly 52 can be programmed (e.g., via either a physical action module 34 incorporated by the circuitry 62 or an operational control module or engine incorporated by the circuitry 62 and communicating with the physical action module 34) to initiate a wakeup operational mode in response to identification of the occurrence of a designated physical action assigned to this control operation (e.g., in response to the patient tapping his or her body (in a region of the sensor component of the implantable sensor 32) three times in succession, the IPG assembly 52 will automatically “wake up” from a sleep mode). With these and other embodiments, for example, the operational control effectuated by the patient’s intentional action can automatically wake up the IPG assembly 52 in preparation for further activities, such as pairing with the external device 36 as described below. In this regard, the operational control effected by detected occurrence of the corresponding, designated physical action can include causing the IPG assembly 52 to automatically transition (or “wake up”) from a sleep mode to a pairing mode or other higher energy active mode. As a point of reference, telemetry, especially modes like Bluetooth® and the like, takes up a lot of energy if left in a high power broadcasting/advertising/sniffing mode and in addition can suffer from fairly long latencies in wake up operational modes. With this in mind, some embodiments of the present disclosure beneficially conserve power by operating the IPG assembly 52 in a lower power sleep mode during periods when no therapy is being provided, and automatically transition to a pairing mode upon detected occurrence of a corresponding, designated physical action, which in turn can mean entering an advertising or sniffing mode in which the IPG assembly 52 attempts to make wireless connections with an external device. Similarly, automatically transitioning from a sleep mode to any other higher energy action mode (e.g., an action mode that requires more power than the sleep mode, but less power than pairing and/or faster connection speed) can beneficially conserve overall power consumption.

In another non-limiting example, the IPG assembly 52 can be programmed (e.g., via either the physical action module 34 incorporated by the circuitry 62 or an operational control module or engine incorporated by the circuitry 62 and communicating with the physical action module 34) to initiate an inactive or sleep operational mode in response to identification of the occurrence of a designated physical action assigned to this control operation (e.g., in response to the patient jumping up and down, the IPG assembly 52 will automatically transition to the inactive mode, thus ceasing any stimulation therapy). With these and related embodiments, a patient can quickly turn the IPG assembly 52 “off” without needing to access a separate remote control.

In another non-limiting example, the IPG assembly 52 can be programmed (e.g., via either the physical action module 34 incorporated by the circuitry 62 or an operational control module or engine incorporated by the circuitry 62 and communicating with the physical action module 34) to pause or extend a current operational mode of the IPG assembly 52 for a predetermined length of time. For example, with some non-limiting embodiments in which the patient therapy system of the present disclosure is implanted and programmed to provide SDB stimulating therapy, the IMD 30 may be programmed to provide stimulation therapy at nighttime, automatically ending the stimulation therapy at a predetermined time of day. In some instances, the patient may wish to extend the therapy beyond the predetermined time (akin to a “snooze” button on an alarm clock); with some of the systems and methods of the present disclosure, the patient can self-extend the provision of therapy beyond the predetermined time by performing the corresponding, designated physical action; the physical action module 34 recognizes that the designated physical action has been performed via information from the implantable sensor 32 and a corresponding operational control routine is performed to automatically extend the provision of stimulation therapy for a predetermined length of time.

In another non-limiting example, the IPG assembly 52 can be programmed (e.g., via either the physical action module 34 incorporated by the circuitry 62 or an operational control module or engine incorporated by the circuitry 62 and communicating with the physical action module 34) to initiate delivery of therapy. For example, with some non-limiting embodiments in which the patient therapy system of the present disclosure is implanted and programmed to provide SDB stimulating therapy, the IMD 30 may be programmed to provide stimulation therapy at a predetermined time of the day and/or after predetermined length of time following minimal or no patient activity. In some instances, the patient may wish to initiate therapy delivery sooner than it otherwise would begin (e.g., the patient is feeling very drowsy and wants to activate a therapy delay override such that therapy “turns on” sooner than it otherwise would). With some of the systems and methods of the present disclosure, the patient can override a therapy delivery delay by performing the corresponding, designated physical action; the physical action module 34 recognizes that the designated physical action has been performed via information from the implantable sensor 32 and a corresponding operational control routine is performed to automatically override a programmed therapy delay and/or initiate provision of stimulation therapy. These optional features may increase patency of the SDB therapy given how quickly some patients become apneic. In related embodiments, where the designated physical action corresponding to the therapy delay override operation entails an action performed on or by the patient’s body (e.g., tapping on the chest), a person other than the patient (e.g., the patient’s spouse) can turn on or activate therapy, or implement other operational control (e.g., adjusting stimulation amplitude) without delay by effecting the designated physical action on the patient, for example while the patient is already sleeping and has already entered apneic.

In yet other embodiments, the control operation or routine implicated by the identified occurrence of a particular, designate physical action can relate to or include interfacing with an external device, such as the external device 36. As a point of reference, the IMD 30 can be configured to interface (e.g., via telemetry) with a variety of external devices. For example, the external device 36 can include, but is not limited to, a patient remote, a physician remote, a clinician portal, a handheld device, a mobile phone, a smart phone, a desktop computer, a laptop computer, a tablet personal computer, etc. The external device 36 can include a smartphone or other type of handheld (or wearable) device that is retained and operated by the patient to whom the IMD 30 is implanted. In another example, the external device 36 can include a personal computer or the like that is operated by a medical caregiver for the patient. The external device 36 can include a computing device designed to remain at the home of the patient or at the office of the caregiver.

With the above in mind, the control operations or routines of the present disclosure can include one or more of confirming, initiating, authenticating, etc., telemetry communications between the IMD 30 and the external device 36. The term “initiating” is intended to encompass both a user action starting at the external device 36, and a user action starting with performance of a designated physical action (e.g., tapping on the chest or other region of the body close to the implanted sensor 32). As a point of reference, telemetry protocols facilitate wireless communication between the IMD 30 and the external device 36. The protocols dictate that the IMD 30 and the external device 36 have to agree on many physical layer and link layer aspects of the data to be exchanged before a successful connection can be established. Rules defining how to establish a connection and perform telemetry communications are referred to as “protocols”. Communication protocols can include or encompass authentication, error detection and correction, and signaling. Communication protocols are implemented in hardware and software, carried for example, by the IMD 30 and the external device 36. Standardized telemetry communication technology or protocol that can be used by one or more entities, in an open source or licensed arrangement. For example, Bluetooth®, Bluetooth® low energy (BLE), near-field magnetic induction (NFMI) communication, Wi-Fi, Zigbee®, etc.

Regardless of the particular telemetry communication protocol employed by the IMD 30 and the external device 36 to communicate, the devices 30, 36 first establish a telemetry “connection” in order to communicate. In telecommunications, a “connection” is the successful completion of the necessary arrangement so that two or more entities (e.g., devices, applications, users of the devices or applications, etc.) can communicate. Two devices can establish a telemetry connection using a telemetry link. A “link” refers to a communications channel that connects two or more communicating devices. This link may be an actual physical link or it may be a logical link that uses one or more actual physical links. In some implementations in which two devices are connected using wireless communications (e.g., telemetry), the link between the respective devices is a logical link. Thus, in various embodiments, the terms “connections” and “link” are used interchangeably.

The IMD 30 can be configured to establish a telemetry connection with the external device 36 upon receiving a connection request from the external device 36. The connection request can generally prompt the IMD 30 to establish a telemetry connection with the external device 36 to allow the external device 36 to program or control operation of the IMD 30 and/or to read data collected from the IMD 30. The particular signal flow for setting up and establishing the telemetry connection can vary depending upon the telemetry protocol employed. In some embodiments, in order for the IMD 30 and the external device 36 to communicate sensitive information between one another (e.g., programming information controlling operation of the IMD 30), the IMD 30 and the external device 36 must establish and employ a “secure” telemetry connection or link. A “secure” telemetry connection or link is a connection or link that is encrypted by one or more security protocols to ensure the securing of data flowing between two or more nodes. For example, in order to initiate a telemetry session with the IMD 30, the external device 36 can send a connection request to the IMD 30 requesting to establish a telemetry connection with the IMD 30. Upon reception of a connection request from the external device 36, an initial telemetry connection can be established between the IMD 30 and the external device 36. The IMD 30 can then determine whether the external device 36 is authorized to communicate with the IMD 30 prior to establishing a secure connection with the external device 36.

In order to establish a secure telemetry connection between devices (e.g., between the IMD 30 and the external device 36), the devices perform a pairing process that establishes or authenticates a secure, trusted relationship between the devices. The pairing process is a mechanism where the devices involved in the communication exchange their identity information to set up trust and get the encryption information needed for future data exchange. The secure, trusted relationship ensures that both devices have authorized telemetry communication between the respective devices and often involve a user/device authorization and/or identification procedures. After two devices have established and authenticated a secure, trusted relationship (also referred to as becoming “paired”), the respective devices can communication with one another, sometimes using the encryption keys previously established between the devices in the pairing process.

Against the above background, the control operations or routines of the present disclosure can include confirming, initiating, or establishing an authenticated communication link between the IMD 30 and the external device 36. For example, the telemetry protocols programmed to the IPG assembly 52 can require that, as part of the pairing process, in order for a secure communication link between the IMD 30 and the external device 36 to be authenticated, a designated physical action must be performed by the patient (and thus recognized or identified by the physical action module 34 based upon information from the implantable sensor 32). The IPG assembly 52 can be programmed (e.g., via either a physical action module 34 incorporated by the circuitry 62 or a telemetry module or engine incorporated by the circuitry 62 and communicating with the physical action module 34) to authenticate an attempted secure communication link between the IMD 30 and the external device 36 only if a designated physical action is identified within a certain time period. By way of non-limiting example, a secure communication link between the IMD 30 and the external device 36 is authenticated only if the implantable sensor 32 detects the patient tapping his or her body (in a region of the sensor component of the implantable sensor 32) a pre-determined number of times as part of the pairing process. In related embodiments, actions performed at the external device 36 can be incorporated into the telemetry protocol. For example, the external device 36 can provide a pairing “button” that a user presses to initiate the pairing process; with these and related embodiments, the telemetry protocols can optionally include a user (e.g., the patient, a clinician, etc.) pressing the pairing button on the external device 36 followed by the patient performing the designated physical action (that is thus sensed by the implantable sensor 32) within a predetermined time period in order for the IMD 30 to authenticate a secure communication link between the IMD 30 and the external device 36.

From the above explanations, it will be understood that some systems and methods of the present disclosure provide enhanced cybersecurity as an addition to, or as an alternative to, conventional or standard cybersecurity methodologies utilized in authenticating a secure telemetry communication link between the IMD 30 and the external device 36. Requiring the patient to perform a designated physical action during pairing can provide an additional layer of authentication when establishing the communication link (e.g., to prevent pairing of the IMD 30 with a malicious device, etc.). For example, the pairing mode can include the IPG assembly 52 periodically broadcasting a unique identifier associated with the IPG assembly 52. This can optionally serve as a privacy mitigation; information is broadcasted only when patient requests via entering pairing mode. A unique identifier is typically required for pairing to function.

In some related, optional embodiments, the programming provided with a telemetry communications module or engine incorporated into the external device 36 can also make reference to or “require” one or more designated physical actions by the patient (or other user) to authenticate a secure communication link with the IMD 30. For example, external device 36 can further include an external sensor akin to the implantable sensor 32. As part of the pairing process or operation, the external device 36 is positioned relative to the patient such that the external sensor can sense the same designated physical action as the implantable sensor 32; a secure communication link between the IMD 30 and the external device 36 is authenticated only if a physical action implicated by the implantable sensor 32 acceptably corresponds with a physical action implicated by the external sensor of the external device 36 (or more simply if the signal from the implantable sensor 32 acceptably corresponds with the signal form the external sensor of the external device 36), thereby ensuring that the correct device is paired to the external device 36 when pairing with the external device 36 is initiated (also referred to as disambiguation; multiple possible pairing devices are available in the vicinity and want to ensure the desired connection is established; also provides a means of confirming pairing to a device and not a false positive on pairing). With these and related embodiments, a physical action (e.g., motion signal) is sensed by both the implantable sensor 32 (and thus the physical action module 34 associated with the IMD 30) and the external device 36. This could be a signal (e.g., a signal generated by the external device 36, an environmental signal, or a patient-executed physical action) that is sensed by both the implantable sensor 32/IMD 30 and the external sensor/external device 36. For example, the external device 36 can be placed on the patient’s body (e.g., chest), followed by the patient tapping on the external device 36, a custom vibration pattern sensed by both the implantable sensor 32/IMD 30 and the external sensor/external device 36, etc. Various algorithms could be employed to determine whether the two signals acceptably correspond with one another. For example, analysis or comparison of peak-to-peak timing of the two signals can be performed; the control operation effected by the IMD 30 would, in some embodiments, only allow pairing with the external device 36 if the external sensor/external device 36 reported the correct timing values (or some other statistic on the signal) along with other authentication information.

Alternatively or in addition, the systems and methods of the present disclosure can utilize programming (e.g., an app) provided with the external device 36 to establish or calibrate parameters of the physical action module 34. For example, as part of a training or calibration mode in which the external device 36 wirelessly communicates with the IMD 30, the external device 36 can prompt the patient to perform a designated physical action (e.g., tapping body, jump, turn left, turn right, raise right arm, raise left arm, etc.). Alternatively or in addition, a series of actions can be utilized for calibration (e.g., the patient is prompted to perform the designated physical action, then lie down for 10 seconds, then stand up for 10 second, then walk 10 steps); this allows the implanted sensor 32 to train and then more accurately sense further activity/posture. During this calibration process, signals generated by the implantable sensor 32 are characterized. In the future, when the implantable sensor 32 detects a similar action, the system 20 performs as assigned operational control as described above. In related embodiments, the external device 36 can be used to program custom, designated physical actions by the patient.

In yet other embodiments, the control operation or routine implicated by the identified occurrence of a particular, designated physical action can relate to or include operation of the implantable sensor 32. For example, in some embodiments the implantable sensor 32 can include an accelerometer sensor component. Accelerometer-based sensors typically provide a wide range of operational modes, each with different power requirements (e.g., a low sampling rate-type operational mode requires less power than a high sampling rate-type operational mode). The implantable sensor 32 can be prompted to operate in a particular mode via logic carried by the implantable sensor 32, by the IPG assembly 52 (for example with embodiments in which the implantable sensor 32 is connected to the IPG assembly 52), or by another device (e.g., the external device 36). Regardless, the implantable sensor 32 can be prompted to automatically initiate or operate in a particular operational mode upon determined occurrence of a corresponding, designated physical action. By way of non-limiting example, the IPG assembly 52 can be programmed (e.g., via either a physical action module 34 incorporated by the circuitry 62 or an operational control module or engine incorporated by the circuitry 62 and communicating with the physical action module 34) to prompt the implantable sensor 32 to operate in a high sampling rate-type operation mode (e.g., appropriate to what the patient is deemed to be doing) in response to identification of the occurrence of a designated physical action assigned to this control operation (e.g., in response to the patient assuming a prone posture, the IPG assembly 52 will automatically cause the implantable sensor 32 to operate in a high sampling rate-type operational mode). With these and other embodiments, for example, power consumption by the implantable sensor 32 can be minimized during time periods in which higher sampling rates are not desired (e.g., the implantable sensor 32 can be run at a very low sample rate (for example 1 Hz) and still “detect” information necessary to deem that a particular designated physical action has occurred (such as posture detection, climbing stairs, running, etc.) that otherwise implicates the need or desire to enter a higher power, higher sampling rate mode (for example 10-100 Hz for respiration detection, 50-500 Hz for cardiac detection, etc.)).

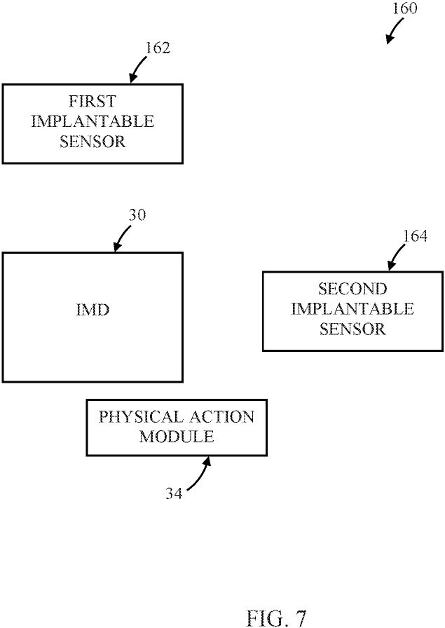

In related embodiments, the operational control of the present disclosure can relate to optional system configurations including two or more redundant implantable sensors. As a point of reference, portions of another patient therapy system 160 in accordance with principles of the present disclosure are schematically reflected in FIG. 7. The system 160 includes the IMD 30 and the physical action module 34 as described above, along with a first implantable motion-based transducer sensor 162 and a second implantable motion-based transducer sensor 164. One or both of the sensors 162, 164 can serve as an input to the physical action module 34 (i.e., as the implantable sensor 32 (FIG. 1) as described above); alternatively or in addition, another implantable sensor (not shown) can be provided that serves as an input source from which the physical action module 34 determines or identifies performance of a designated physical action by the patient as described above. While shown apart from the IMD 30, one or both of the sensors 162, 164 can be carried within a structure of the IMD 30 (e.g., such as within the housing 60 as with the embodiments of FIG. 6). Regardless, the implantable motion-based transducer sensors 162, 164 each include or carry a motion-based transducer sensor component (e.g., accelerometer, gyroscope, etc.) and are operable as redundant sensors. While two of the redundant sensors 162, 164 are shown, in other embodiments, three or more redundant sensors can be provided.

The system 160 can be programmed to operate the redundant motion-based transducer sensors 162, 164 (e.g., redundant accelerometers, redundant gyroscopes) in various manners. In some embodiments, the first motion-based transducer sensor 162 is dedicated to sensing a parameter indicative of a particular designated physical action, whereas the second motion-based transducer sensor 164 serves other purposes. With these and similar embodiments, the first motion-based transducer sensor 162 can be operated as a low resolution, low sample rate, low power consumption sensor (e.g., accelerometer) that “handles” one particular designated physical action and corresponding operational control (e.g., the “wake up” control operations described above), while the second sensor 164 can be operated in other modes to serve other purposes. In some embodiments, the redundant motion-based transducer sensors 162, 164 can be specifically tuned to provide necessary information for the physical action module 34 to detect occurrence of a particular designated physical action (e.g., by the filter parameters used, the orientation of the sensor chip relative to the IMD 30/patient, the type of motion-based transducer sensor employed, etc.). Regardless, the control operations of the present disclosure can include switching between the redundant sensors 162, 164. For example, the first motion-based transducer sensor 162 can be a 3-axis accelerometer arranged, following implant, with the major axis of the patient, and the second motion-based transducer sensor 164 is a 3-axis accelerometer arranged, following implant, at 45 degrees relative to the first sensor 162; with this in mind, the designated physical action can be a posture change from lying down to a more upright posture and the corresponding control operation can be switching the input to a patient monitoring module from the first accelerometer sensor 162 to the second accelerometer sensor 164 (e.g., under these circumstances, the second accelerometer sensor 164 (positioned at 45 degrees relative to the first accelerometer sensor 162) is better able to detect physiological signals (e.g., heart rate, respiration, etc.) than the first accelerometer sensor 162).

Returning to FIGS. 1-6, in some non-limiting embodiments the IPG assembly 52 can be programmed (e.g., via either the physical action module 34 incorporated by the circuitry 62 or an operational control module or engine incorporated by the circuitry 62 and communicating with the physical action module 34) to prompt electronic storage of sensed physiologic information recorded within a predetermined time period (e.g., 1 minute) before and/or after the time of the sensed performance of the designated physical action. This stored information (e.g., posture, ECG, EEG, cardiac) during an episode (e.g., awakening, cardiac arrhythmia, seizure, etc.) could be available for later review by a clinician.

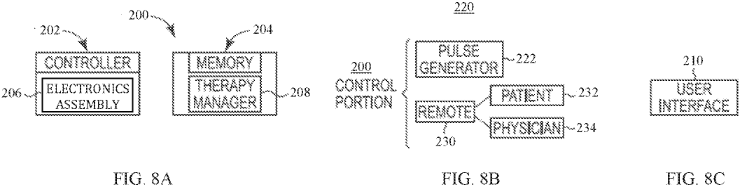

As implicated by the above descriptions, one or both of the IMD 30 and the external device 36 includes a controller, control unit, or control portion that prompts performance of designated actions. FIG. 8A is a block diagram schematically representing a control portion 200, according to one example of the present disclosure. In some examples, the control portion 200 includes a controller 202 and a memory 204. In some examples, the control portion 200 provides one example implementations of a control portion forming a part of, implementing, and/or managing any one of devices, systems, assemblies, circuitry, managers, engines, functions, parameters, sensors, electrodes, modules, and/or methods, as represented throughout the present disclosure in association with FIGS. 1-7.

In general terms, the controller 202 of the control portion 200 comprises an electronics assembly 206 (e.g., at least one processor, microprocessor, integrated circuits and logic, etc.) and associated memories or storage devices. The controller 202 is electrically couplable to, and in communication with, the memory 204 to generate control signals to direct operation of at least some the devices, systems, assemblies, circuitry, managers, modules, engines, functions, parameters, sensors, electrodes, and/or methods, as represented throughout the present disclosure (e.g., the physical action module 34 (FIG. 1) can be a software program stored on a storage device, loaded onto the memory 204, and executed by the electronics assembly 206). In addition, in some examples these generated control signals include, but are not limited to, employing therapy manager 208 stored in the memory 204 to at least manage therapy delivered to the patient, for example therapy for sleep disordered breathing, and/or manage and operate designated physical action sensing in the manner described in at least some examples of the present disclosure. It will be further understood that the control portion 200 (or another control portion) may be employed to operate general functions of the various therapy devices/systems described throughout the present disclosure.

In response to or based upon commands received via a user interface (e.g. user interface 210 in FIG. 8C) and/or via machine readable instructions, the controller 202 generates control signals to implement therapy implementation, therapy monitoring, therapy management, and/or management and operation of designated physical action sensing and control in accordance with at least some of the previously described examples of the present disclosure. In some examples, the controller 202 is embodied in a general purpose computing device while in some examples, the controller 202 is incorporated into or associated with at least some of the associated devices, systems, assemblies, circuitry, sensors, electrodes, components of the devices and/or managers, engines, parameters, functions etc. described throughout the present disclosure.

For purposes of this application, in reference to the controller 202, with embodiments in which the electronics assembly 206 comprises or includes at least one processor, the term “processor” shall mean a presently developed or future developed processor (or processing resource) or microprocessor that executes sequences of machine readable instructions contained in a memory. In some examples, execution of the sequences of machine readable instructions, such as those provided via the memory 204 of control portion 200 cause the processor to perform actions, such as operating the controller 202 to implement sleep disordered breathing (SDS) therapy and related management and/or management and operation of designated physical action sensing, as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., nontransitory tangible medium or non-volatile tangible medium, as represented by the memory 204. In some examples, the memory 204 comprises a computer readable tangible medium providing non-volatile storage of the machine readable instructions executable by a process of the controller 202. In other examples, hard wired circuitry may be used in place of or in combination with machine readable instructions to implement the functions described. For example, the electronics assembly 206 may be embodied as part of at least one application-specific integrated circuit (ASIC), at least one integrated circuit, a microprocessor and ASIC, etc. In some examples, the controller 202 is not limited to any specific combination of hardware circuitry and machine readable instructions, nor limited to any particular source for the machine readable instructions executed by the controller 202.

FIG. 8B is a diagram 220 schematically illustrating at least some manners in which the control portion 200 can be implemented, according to one example of the present disclosure. In some examples, the control portion 200 is entirely implemented within or by an IPG assembly 222, which has at least some of substantially the same features and attributes as the IPG assembly 52 as previously described in association with at least FIG. 2. In some examples, the control portion 200 is entirely implemented within or by a remote control 230 (e.g. a programmer) external to the patient’s body, such as a patient control 232 and/or a physician control 234. In some embodiments, the remote control 230 is akin to the external device 36 (FIG. 1) described above. In some examples, the control portion 200 is partially implemented in the IPG assembly 222 and partially implemented in the remote control 230 (at least one of the patient control 232 and the physician control 234). In some examples the control portion 200 may be implemented via a server accessible via the cloud and/or other network pathways. In some examples, the control portion 200 may be distributed or apportioned among multiple devices or resources such as among a server, an IMD, and/or a user interface

In some examples, in association with the control portion 200, the user interface (210 in FIG. 8C) is implemented in the remote control 230. FIG. 8C is a block diagram schematically representing the user interface 210, according to one example of the present disclosure. In some examples, the user interface 210 forms part or and/or is accessible via a device external to the patient and by which the therapy system may be at least partially controlled and/or monitored. The external device hosting the user interface 210 may be a patient remote (e.g., 232 in FIG. 8B, for example a smartphone operating a custom software application), a physician remote (e.g., 234 in FIG. 8B) and/or a clinician portal. In some examples, the user interface 210 comprises a user interface or other display that provides for the simultaneous display, activation, and/or operation of at least some of the various systems, assemblies, circuitry, engines, sensors, components, modules, functions, parameters, as described in association with FIGS. 1-7. In some examples, at least some portions or aspects of the user interface 210 are provided via a graphical user interface (GUI), and may comprise a display and input.

Returning to FIG. 1, information from the implantable sensor 32, including the motion-based transducer sensor component, can optionally utilized to sense or detect other parameters associated with the patient, that may or may not include involuntary actions. Moreover, in some embodiments, the IMD 30 can be controlled to operate in response to involuntary actions by the patient as sensed by the implantable sensor 32. Non-limiting examples of some possible control features implemented by the systems and methods of the present disclosure can comprise at least some of substantially the same features and attributes as described within at least U.S. Application Serial No. 16/092,384, filed Oct. 9, 2018 and entitled “ACCELEROMETER-BASED SENSING FOR SLEEP DISORDERED BREATHING (SDB) CARE”, the entire teachings of which are incorporated herein by reference. In related embodiments, the systems of the present disclosure can include one or more additional implantable sensors (in addition to the implantable sensor 32). Information signaled by the one or more additional implantable sensors can optionally be employed (along with information from the implantable sensor 32) as part of the recognition or identification of an occurrence of a designated physical action as described above. Alternatively or in an addition, the information signaled by the one or more additional implantable sensors can be employed in monitoring the patient, formulating a therapy regimen, etc., as described, for example, in U.S. Application Serial No. 16/092,384.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1-60. (canceled)

61. A method comprising:
sensing intentional performance by a patient of a designated physical action via an implantable sensor implanted within the patient; and
controlling operation of an implantable medical device system based upon the sensed performance of the designated physical action, the implantable medical device system including an implantable medical device implanted within the patient.

62. The method of claim 61, wherein the step of controlling includes altering an operational mode of the implantable medical device.

63. The method of claim 61, wherein the implantable medical device system further includes an external device located outside the patient and configured to wirelessly communicate with the implantable medical device, and further wherein the step of controlling includes initiating an authenticated communication link between the implantable medical device and the external device.

64. The method of claim 63, wherein the external device is selected from the group consisting of a smart phone, a tablet computer, a laptop computer, a desktop computer, a remote control, a clinical programmer, and a communication bridge.

65. The method of claim 63, wherein the step of initiating is performed as part of a pairing process between the implantable medical device and the external device.

66. The method of claim 65, wherein the pairing process establishes a secure communication link between the implantable medical device and the external device.

67. The method of claim 63, further comprising:
operating the external device to initiate a pairing process between the external device and the implantable medical device; and
completing the pairing process only if the step of sensing corresponds with the step of operating the external device to initiate the pairing process.

68. The method of claim 63, further comprising:
positioning the external device proximate the sensor; and
sensing the intentional performance by the patient of the designated physical action via an external sensor carried by the external device simultaneously with the step of sensing via the implantable sensor.

69. The method of claim 61, further comprising:
operating the implantable medical device to treat sleep disordered breathing.

70. The method of claim 61, wherein the implantable sensor includes a motion-based transducer sensor component.

71. The method of claim 61, wherein the designated physical action includes tapping on a body of the patient in a region of a sensor component of the implantable sensor.

72. The method of claim 61, wherein the implantable medical device system is programmed to recognize a plurality of different designated physical actions, and further wherein the implantable sensor generates a signal indicative of a sensed physical action characteristic of the patient, the method further comprising:
receiving a signal from the implantable sensor; and
recognizing performance of a current designated physical action from the plurality of different designated physical actions based upon the signal.

73. The method of claim 72, wherein the implantable medical device system is programmed to perform a different control operation in response to each of the plurality of different designated physical actions.

74. A system for treating a patient comprising:
an implantable medical device system including an implantable medical device; and
an implantable sensor configured to at least sense a characteristic indicative of intentional performance by the patient of a designated physical action;
wherein the implantable medical device system is programmed to control operation of the implantable medical device system based upon the sensed performance of the designated physical action.

75. The system of claim 74, further comprising:
an external device configured to be operable as part of the system from outside the patient.

76. The system of claim 75, wherein the designated physical action includes a signal generated by the external device.

77. The system of claim 75, wherein the implantable medical device and the external device are configured to wirelessly communicate, the system further comprising:
a telemetry communications module programmed to perform telemetry protocol instructions for establishing an authenticated communication link between the implantable medical device and the external device;
wherein the telemetry protocol instructions include identification of an occurrence of the designated physical action.

78. The system of claim 74, wherein the system is programmed to prompt the implantable medical device to switch from a sleep operational mode to a pairing operational mode in response to the sensed performance of the designated physical action.

79. The system of claim 74, wherein the implantable sensor includes a motion-based transducer sensor component.

80. The method of claim 79, wherein the motion-based transducer sensor includes a component selected from the group consisting of an accelerometer, a gyroscope, and a pressure sensor.
